(19) United States
(12) Patent Application Publication
(10) Pub. No.: US 2024/0100353 A1
(43) Pub. Date: Mar. 28, 2024

(54) Light Therapy Device With Neckband for Power Supply

(71) Applicants: Steven Yu, Fairfax, VA (US); Jianheng Dong, Shenzhen (CN)

(72) Inventors: Steven Yu, Fairfax, VA (US); Jianheng Dong, Shenzhen (CN)

(21) Appl. No.: 18/472,409

(22) Filed: Sep. 22, 2023

Related U.S. Application Data
(60) Provisional application No. 63/377,010, filed on Sep. 24, 2022.
(60) Provisional application No. 63/478,978, filed on Jan. 8, 2023.
(60) Provisional application No. 63/502,561, filed on May 16, 2023.

(51) Int. Cl. A61N 5/06

(52) U.S. Cl. CPC A61N 5/0616; A61N 2005/0645; A61N 2005/0651; A61N 5/067

(57) ABSTRACT

A light therapy device for treating a user's skin (such as wrinkles or acne). The device comprises a neckband for wearing on the user's neck and a light-emitting appliance comprising light diodes for delivering light therapy. Example formats for the light-emitting appliance include facemasks, eyemasks, and helmets. The device further comprises a power supply that may be contained within the neckband or carried by the neckband. An external electrical wire is for connecting the light-emitting appliance to the power supply. The neckband could have a variety of different configurations. Also disclosed are skin therapy kits and methods for light therapy to a user's skin.

### TECHNICAL FIELD

This invention relates to light therapy to treat skin conditions.

### BACKGROUND

Various skin conditions (e.g. acne or aging skin such as wrinkles) can be treated by light therapy. Existing devices are available for performing light therapy. Such light therapy devices have a variety of different formats such as facemasks, eyemasks, skin pads, helmets, etc. FIG. 1 shows an example of an existing light therapy device. It has a facemask 160 with light-emitting diodes (LED) on the inside, a battery/controller case 164, and a power supply wire 162 connecting the facemask 160 to battery/controller case 164. The user wears facemask 160 and activates the device for light therapy treatment to the face. However, battery case 164 is not convenient to carry. For example, the user must carry battery case 164 in their hand, lay it on a table, or put it in their pants pocket. Thus, there is a need for a more convenient way to carry the power supply for the device.

### SUMMARY

One aspect of this invention is a light therapy device comprising a neckband for wearing on a user's neck and a light-emitting appliance. The light therapy device further comprises a power supply that may be contained within the neckband or carried by the neckband; and an external electrical wire for connecting the light-emitting appliance to the power supply. Having the neckband hold or carry the power supply and be separate from the light-emitting appliance could have various benefits such as more convenient carrying or portability, lighter weight for wearing on the face, improved comfort, more design flexibility, less bulk, etc.

Light-Emitting Appliance. The light-emitting appliance is for applying to a part of the user's head (e.g. for attaching to face, wearing on face, or wearing on scalp, etc.). The light-emitting appliance comprises light diodes for delivering light therapy to the treatment site. Examples of light diodes that could be used include light-emitting diodes (LED) and laser diodes. The light-emitting appliance could be any suitable format for wearing by the user on any part of the head, such as face, forehead, or scalp. Examples formats for the light-emitting appliance include face mounting bases (e.g. skin pads), eyemask, eyewear frame (e.g. glasses or goggles), facemask, or headgear worn on the top of the head (scalp area), such as helmet, hat, cap, hood, etc. (for hair growth treatment). The light-emitting appliance could have any suitable feature for applying to the user's head, such as straps, sidearms, adhesives, etc.

Power Supply. The power supply provides electrical power for the device and could be contained within or carried by the neckband. The power supply could be any suitable source for providing electrical power. For example, the power supply could be one or more batteries (e.g. conventional alkaline battery, rechargeable battery, etc.). In another example, the power supply could be a battery compartment (which holds the battery(s) that may or may not be provided with the device). In some embodiments, the neckband comprises the power supply. For example, the power supply could be inside the neckband body or a part thereof. In some embodiments, the neckband is part of a neckband assembly that comprises the neckband and the power supply is carried by the neckband. That is, the light therapy device comprises a neckband assembly, which comprises the neckband and the power supply carried by the neckband. For example, the power supply could be a battery pod that is attached to or connected to the neckband. As the power supply, the battery pod comprises a battery or battery compartment for holding a battery. The battery pod could also function as a controller for the light therapy device.

Electrical Wire. The device comprises one or more electrical wires for connecting the light-emitting appliance to the power supply. The electrical wire travels externally between the light-emitting appliance and the power supply. The electrical wire may be permanently connected to the light-emitting appliance, or the power supply, or both. The electrical wire may be detachable from the light-emitting appliance, or the power supply, or both. The electrical wire may be a separate and detached item; or may be provided already connected to the power supply, or the light-emitting appliance, or both.

The electrical wire could be any suitable length. Because the distance between the neckband and the light-emitting appliance on the user's head is relatively close, the electrical wire could be relatively short in length. Moreover, having a wire length that is too long could cause drooping, tangling, or other inconvenience. For example, the wire length could be 75 cm or 60 cm; or in the range of 20-75 cm, or in the range of 20-60 cm.

Neckband. The neckband could have any suitable shape for fitting on the neck, such as having a shape like an arc, curve, semicircle, or loop. The neckband fits on the user's neck but does not necessarily touch the user's neck. For example, parts of the neckband may rest on the user's shoulders. The neckband could be a single body component or could comprise multiple components.

In some embodiments, the neckband has a fixed configuration. For example, the neckband could be rigid or minimally flexible. In some embodiments, the neckband has an open loop configuration (i.e. not a fully closed loop). For example, the neckband could have an arc, curve, or semicircle shape. In some embodiments, the neckband has a permanent closed loop configuration.

Open/Closed Configurations. In some embodiments, the neckband has an open configuration and a closed configuration. The open configuration is for allowing the user to wear or remove the neckband. The closed configuration is for allowing the neckband to hang from the user's neck. There could be more than one open configuration or more than one closed configuration (e.g. there could be a range of open or closed configurations). The open and closed configurations could be formed in any suitable way. For example, the neckband could have a releasable fastening mechanism that is engaged to form the closed configuration and is disengaged to form the open configuration. Examples of releasable fastener mechanisms include clasps, hooks, lobster claws, magnets, clips, rings, pins, hook-and-loop, etc.

In embodiments for a neckband assembly comprising a neckband and a battery pod carried by the neckband, the closed and open configurations could be formed by a free end of the neckband meeting with the battery pod (such as joining, connecting, locking, intersecting, overlapping, etc.) and separating from the battery pod. The neckband assembly could comprise a releasable fastener mechanism (see examples above) for meeting/separating of the neckband to the battery pod.

Two Arms. In some embodiments, the neckband comprises two arms that meet each other (such as joining, connecting, locking, intersecting, overlapping, etc.) to form a closed configuration and separate from each other to form an open configuration. For example, the two arms could meet and separate by a releasable fastener mechanism (see examples above). The fastener mechanism could comprise a first fastener part on the first arm and a second fastener part (counterpart) on the second arm. As an example, the first fastener part and the second fastener part could be magnets or ring-and-clasp.

In another example, the two arms could be elastically flexible with a natural bend that deflects inward when in relaxed condition. In closed configuration, the two arms meet each other. In open configuration, the two arms are pulled apart against the natural bend and the two arms do not meet. As an example, the two arms could overlap or intersect in closed configuration, but not overlap or intersect in open configuration. The elastic flexibility may be a property of the arms themselves or may be imparted by another component of the neckband assembly (e.g. spring or rubber connector). A neckband assembly comprising the two elastically flexible arms could further comprise a spring (e.g. coil spring, leaf spring, etc.) attached to one or more of the arms to provide or enhance elastic flexibility.

Alternatively, it is not necessary that the two elastically flexible arms must be in completely closed configuration to operate. Instead of open/closed configuration, this neckband could be defined as having a wide gap configuration and a narrow gap configuration. The gap is the distance between the upper ends of the two arms. In the wide gap configuration, the gap is sufficiently wide to allow the user to wear or remove the neckband. In the narrow gap configuration, the gap is sufficiently narrow to allow the neckband to hang from the user's neck. Thus, the narrow gap configuration has a smaller gap than the wide gap configuration.

Alternatively, instead of open/closed configurations, the neckband could be defined as having a flexed-in configuration (arms deflect inward) and a flexed-out configuration (arms are flexed outward). In the flexed-out configuration, the two arms are spread apart to allow the user to wear or remove the neckband. In the flexed-in configuration, the arms are allowed to naturally deflect inward so that the neckband can hang from the user's neck.

Miscellaneous. The following may be further distinguishing characteristics of the light therapy device. The neckband of this invention is not a light therapy neck collar or a light-emitting appliance for the neck. As such, one or more of the following may be a characteristic of the neckband: it is not part of the light-emitting appliance; or it is not directly attached to the light-emitting appliance; or it does not provide light therapy to the neck; or it does not provide light therapy to the treatment site; or it is not a flat folding panel; or it does not have any light diodes (for skin treatment); or it is not electrically powered; or is not directly connected to the external electric wire; or it is not attached to the power supply or connected thereto by an electric wire; or it does not have any light diodes (for skin treatment) along its inner perimeter side.

In a neckband assembly, the battery pod could be directly attached to the neckband or attached via a connecting piece (e.g. hinge, ring, etc.), wherein the connecting piece is not an external electrical wire. As more examples, one or more of the following may be a characteristic of the device of this invention: the power supply is directly connected to the light-emitting appliance; or the power supply (e.g. battery pod) does not provide electric power to the neckband.

As more examples, one or more of the following may be a characteristic of the device of this invention: it is not controlled by wireless remote control (e.g. Bluetooth or Wi-Fi); or it does not have an audio microphone; or it does not have an audio output speaker (including miniature); or it does not have a radio communication receiver, transmitter, or transceiver (e.g. Bluetooth or Wi-Fi); or it does not have any wireless remote control receiver that controls the operation of the device (e.g. radio, infrared, etc.); or it does not communicate with an external personal electronic device such as a smartphone.

Light Therapy Kit. Another aspect of this invention is a skin therapy kit that comprises a light therapy device described herein. The components of the skin therapy kit are provided together in the same package. The various components of the light therapy device may be provided as separate parts or come pre-assembled (partially or fully). The kit comprises the neckband and a light-emitting appliance as described herein.

If provided as a distinct component of the device, the kit could further comprise a power supply. The kit could comprise a battery pod, which may be pre-assembled with the neckband (as a neckband assembly) or a separate component (e.g. to be assembled by the user). The kit could further comprise one or more electrical wires for connecting the light-emitting appliance to the power supply. The electrical wire could be already connected to the light-emitting appliance, or power supply, or both; or be a separate component that is attachable and detachable therefrom.

For relevant device embodiments, the kit could further comprise multiple (two or more) adhesive pieces (such as strips, slabs, beds, patches, pads, etc). The kit could further comprise one or more batteries for the power supply. For device embodiments that use a rechargeable battery, the kit could further comprise accessories for recharging, such as a charging block, wire connector, charging plug adapter, etc.

The kit could further comprise an oral nutritional supplement or topical cosmetic formulation that work synergistically with the light therapy. The oral nutritional supplement comprises one or more cosmetic or therapeutic ingredients, such as collagen peptides (from hydrolyzed collagen proteins), hyaluronic acid, etc. The topical cosmetic formulation comprises one or more cosmetic or therapeutic ingredients, such as collagen peptides (from hydrolyzed collagen proteins), hyaluronic acid, vitamin C, vitamin E, etc. The topical cosmetic formulation may be a serum, lotion, gel, paste, cream, ointment, or other type of topical skin formulation. The oral nutritional supplement or topical cosmetic formulation may be provided in a container such as a bottle, jar, can, squeeze tube, vial, etc.

Treatment Method. Another aspect of this invention is a method of light therapy to a user's skin. Examples of skin areas that could be treated include face, forehead, or scalp. The light therapy device could be used for treating a variety of medical or cosmetic skin conditions such as aging, eczema, acne, psoriasis, sun damage, wrinkles or fine lines, color blemishes, inflammation, under-eye dark circles, hair loss on scalp, etc.

The user could perform one or more of the following steps. Having a light therapy device as described herein. Wearing the neckband on the neck. Applying the light-emitting appliance to the desired treatment site on the user's head (e.g. attach to face, strap onto face, wear over scalp, etc.). Activating the device to cause the light-emitting appliance to emit therapeutic light. Performing the light therapy treatment for a sufficient duration. For example, the duration of treatment could be in the range of 2-60 minutes for each session (e.g. 10 minutes for each session). The treatment could be applied for a single or multiple sessions per day. The external electrical wire may already connect the power supply to the light-emitting appliance. Otherwise, the method could further comprise connecting the electrical wire to the power supply, or the light-emitting appliance, or both.

For a neckband having open and closed configurations, the method could comprise one or more of the following steps. Having the neckband in open configuration. The neckband may already be in open configuration or the user may perform opening the neckband. Wearing the neckband on the neck. Closing the neckband into closed configuration. Performing the light therapy treatment. Opening the neckband into open configuration. Removing the neckband.

For a neckband with a releasable fastener mechanism, the method could comprise one or more of the following steps. Having the fastener mechanism disengaged. The fastener mechanism may already be disengaged or the user may perform the step of disengaging the fastener mechanism. Wearing the neckband on the neck. Engaging the fastener mechanism to close the neckband. Performing the light therapy treatment. Disengaging the fastener mechanism to open the neckband. Removing the neckband. For a neckband with two arms that meet by a releasable fastener mechanism: engaging the fastener mechanism to close the neckband; disengaging the fastener mechanism to separate the two arms (into open configuration).

For a neckband with two elastically flexible arms, the method could comprise one or more of the following steps. Pulling the arms apart to open the neckband. Wearing the neckband on the neck. Releasing the two arms so that the neckband hangs on the user's neck. Performing the light therapy treatment. Pulling the arms apart to open the neckband. Removing the neckband. In some cases, releasing the two arms causes the arms to meet and the neckband forms a closed configuration. For a neckband having narrow and wide gap configurations: pulling apart the arms opens the neckband into wide gap configuration so that the user can wear the neckband; releasing the two arms causes the neckband to form the narrow gap configuration so that the neckband hangs from the neck; for removing the neckband, pulling the arms apart again to form the wide gap configuration.

For a neckband having flexed-in and flexed-out configurations: pulling apart the arms opens the neckband into flexed-out configuration so that the user can wear the neckband; releasing the two arms causes the neckband to form the flexed-in configuration so that the neckband hangs from the neck; for removing the neckband, pulling the arms apart again to form the flexed-out configuration.

### BRIEF DESCRIPTION OF THE DRAWINGS

FIG. 1 shows an example of a prior art light therapy facemask device.

FIGS. 2A-2C show an example device of this invention.

FIG. 3 shows a light therapy device worn by a user.

FIG. 4 shows another example device of this invention.

FIGS. 5A and 5B show another example device of this invention.

FIGS. 6A and 6B show another example neckband assembly for a device of this invention.

FIGS. 7A-7D show another example neckband assembly for a device of this invention.

FIG. 8 shows another example neckband assembly for a device of this invention.

FIG. 9 shows another example neckband assembly for a device of this invention.

FIGS. 10A and 10B demonstrate how inner perimeter length is measured for representative neckbands.

FIGS. 11A-11C demonstrate how maximum body width is measured for representative neckbands.

FIGS. 12A and 12B show an example eyemask as a light-emitting appliance.

FIG. 13 show an example eyewear frame as a light-emitting appliance.

FIGS. 14A and 14B show an example full facemask as a light-emitting appliance.

FIGS. 15A and 15B show an example helmet as a light-emitting appliance.

FIG. 16 shows an example skin therapy kit of this invention.

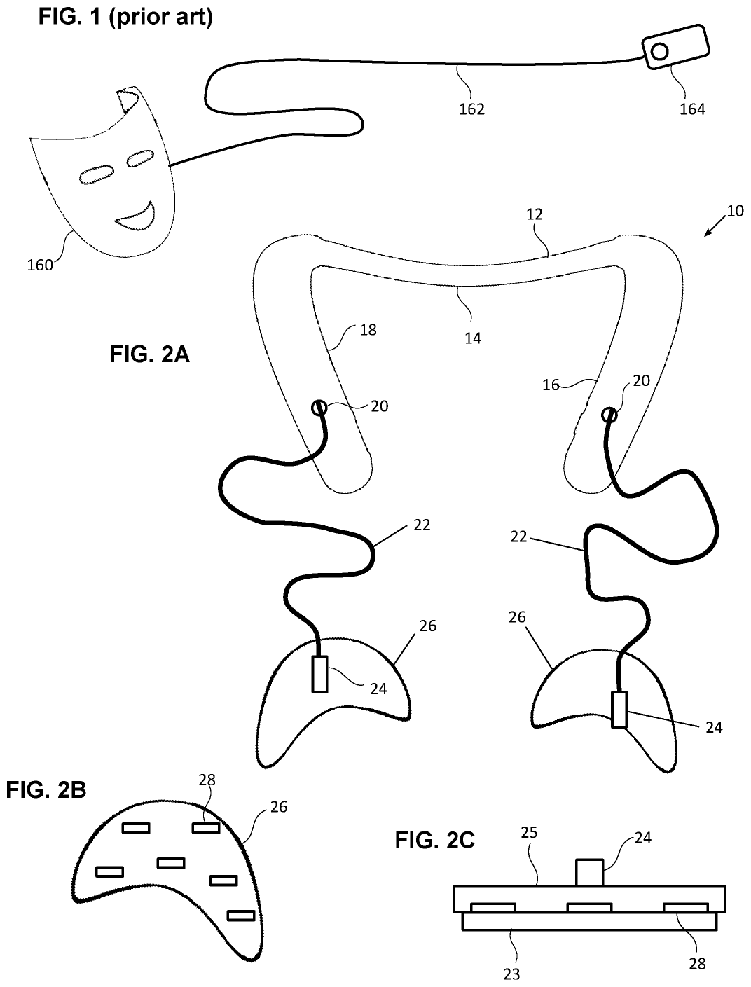

### DETAILED DESCRIPTION OF EXAMPLE EMBODIMENTS

Drawings are provided to help understand the invention and illustrate examples of specific embodiments of the invention. The drawings herein are not necessarily made to scale or actual proportions. For example, the size of components may be adjusted to accommodate the page size.

FIGS. 2A-2C show one example for the light therapy device. FIG. 2A shows an overall view of light therapy device 10, which has a neckband 12 of a general arc shape. Neckband 12 has left arm 16 and right arm 18 that are connected by an arch bridge 14. Left and right are designated from the perspective of the user wearing neckband 12. Towards the end of arms 16 and 18, there are two exit holes 20 in neckband 12 through which electrical wires 22 exit out. Wires 22 are connected to two skin pads 26, which deliver light therapy to the target site on the facial skin. This view shows the outward facing side of skin pads 26, which has wire connectors 24 for attachment to wires 22. Neckband 12 is made of hard plastic and has a rigid, non-flexible shape.

FIG. 2B shows the skin-facing (back) side of skin pads 26. On this side, there are a series of LED lights 28 that emit therapeutic light towards the skin. There is also a skin adhesive on this side of the skin pad. FIG. 2C is a side view of skin pad 26 that better shows adhesive layer 23 and support base 25, which is the main body of skin pad 26. Skin pads 26 could be manually detachable from wires 22. This could be beneficial to facilitate easier use or allow replacement of skin pads 26 if adhesive layer 23 loses adhesiveness. Alternately, skin pads 26 could use a replaceable adhesive layer, such as replaceable (e.g. peel off) adhesive strips or patches.

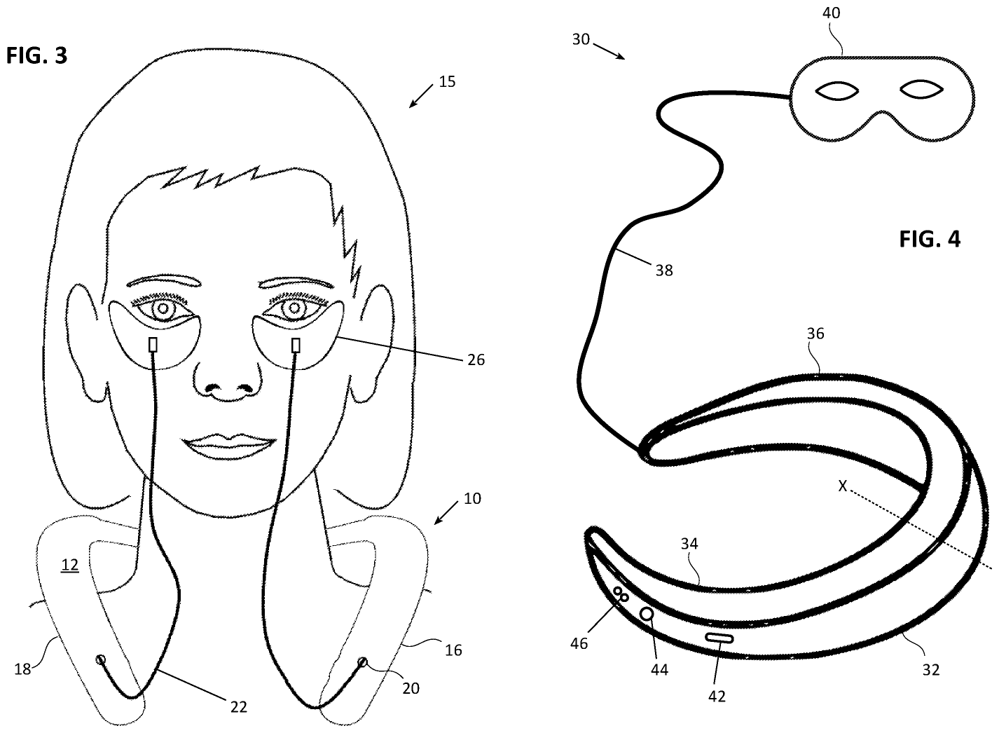

FIG. 3 shows light therapy device 10 worn by a user 15. Neckband 12 fits around the user's neck with arch bridge 14 at the back of the neck and arms 16, 18 extending forward over the shoulders. User 15 has undereye dark circles that she wants treated. Skin pads 26 are adhered to the target site of the user's face. Skin pads 26 emit red or near-infrared (IR) light for treatment of the undereye skin.

FIG. 4 shows another example for the light therapy device. This shows an overall view of light therapy device 30 with a perspective view of an arc-shaped neckband 32 component. Neckband 32 contains a rechargeable battery inside. Neckband 32 has left arm 34 and right arm 36. An electrical wire 38 is connected to arm 36 and supplies power from the battery inside neckband 32. Wire 38 is also connected to an eyemask 40 (as the light-emitting appliance), which delivers light therapy to the skin around the eyes. On left arm 34 of neckband 32, there is a power switch 42, an activation light 44 indicating the on/off status of device 30, and two small indicator lights 46. Device 30 is activated and deactivated by pressing power switch 42. Right arm 36 of the neckband 32 has an electrical charging port (not shown in this view) for recharging. Neckband 32 is made of hard plastic and has a rigid, non-flexible shape.

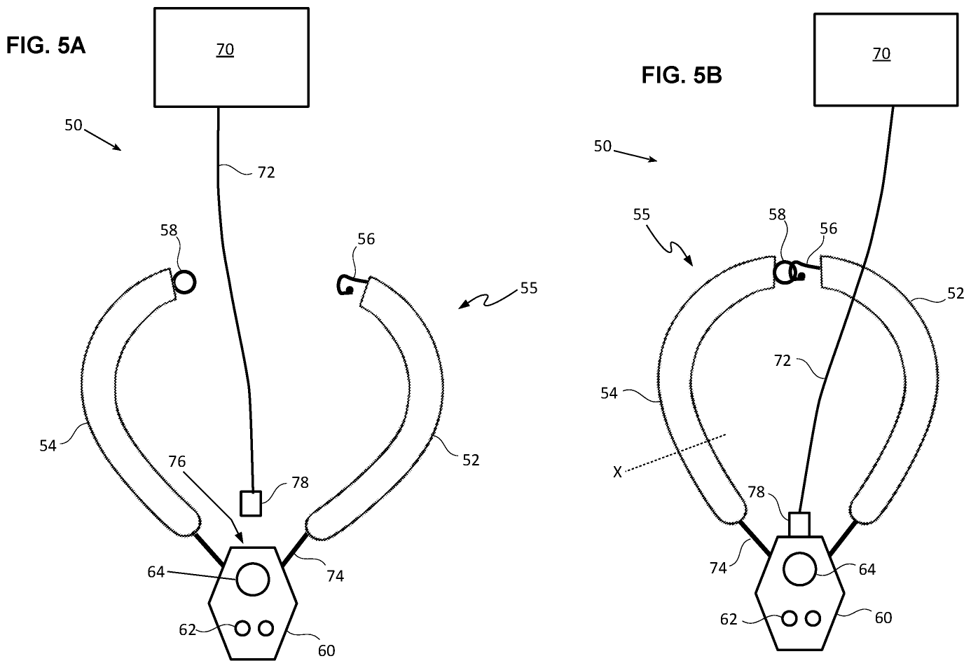

FIGS. 5A and 5B show another example for the light therapy device in which the neckband has a closed configuration and an open configuration using a releasable fastener mechanism. FIG. 5A shows neckband 55 of device 50 in open configuration. Neckband 55 is part of a neckband assembly that further comprises a battery pod 60. Neckband 55 has left arm 52 with a hook 56 at its upper end, and right arm 54 with a ring 58 at its upper end. Here, hook 56 and ring 58 fastener mechanism is disengaged and arms 52, 54 are separated to facilitate wearing (or removing) around the neck. Battery pod 60 hangs from neckband 55 like a pendant. Battery pod 85 comprises a battery compartment for holding a battery (e.g. AAA-size battery or rechargeable battery) and providing user controls. Battery(s) may or may not be supplied with device 50. For example, the user may provide the battery (e.g. AAA-size battery) separately.

Battery pod 60 is attached to each arm 52, 54 by a suspension link 74 that acts as a swivel connection. Having a swivel connection could be useful for more convenient user handling. Battery pod 60 has an activation switch 64 and indicator lights 62. An electrical wire 72 is connected to a light-emitting appliance, which is represented as component block 70. A wire plug 78 is at the other end of wire 72. The user inserts plug 78 into a wire port 76 located at the top of battery pod 60 to supply electrical power to light-emitting appliance 70.

FIG. 5B shows neckband 55 in closed configuration and wire plug 78 is inserted into wire port 76. Arms 52 and 54 are joined by hook 56 and ring 58 fastener mechanism. With wire 72 connected, pressing activation switch 64 activates light-emitting appliance 70. After the treatment session, the user removes neckband 55 by detaching hook 56 from ring 58 and separating arms 52, 54. Also, wire 72 could be unplugged for more convenient storage.

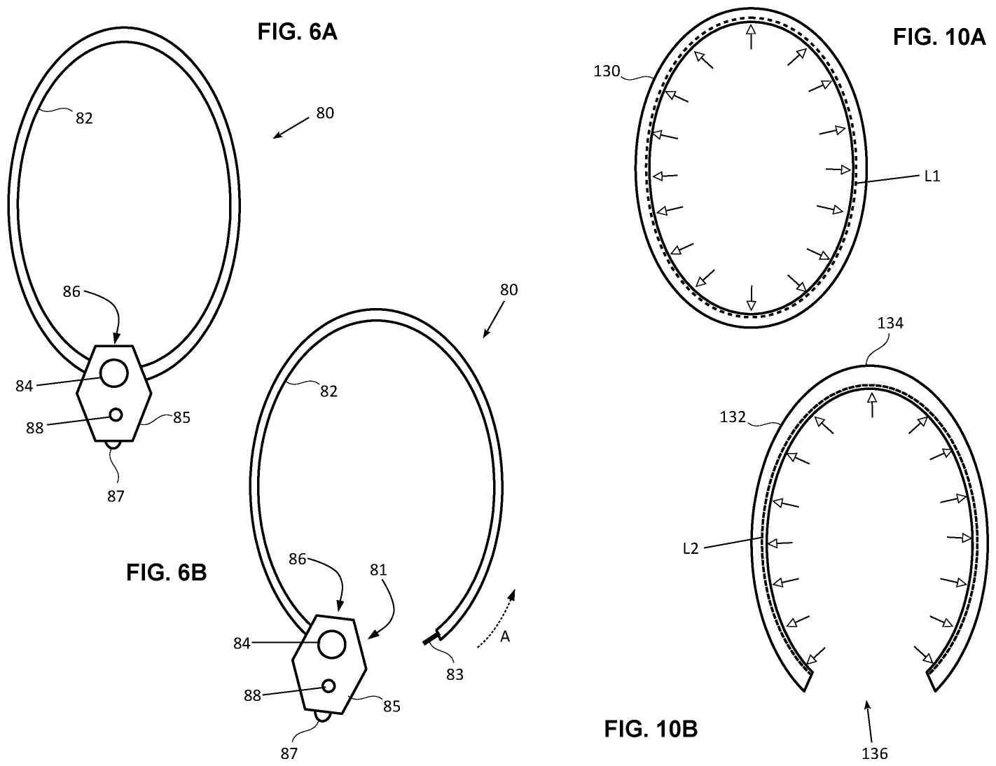

FIGS. 6A and 6B show another example of a neckband assembly for the light therapy device using a releasable fastener mechanism. Neckband assembly 80 comprises a loop neckband 82 that has a closed configuration and an open configuration. FIG. 6A shows neckband 82 in closed configuration. Neckband assembly 80 further comprises a battery pod 85 that is attached to loop neckband 82. Battery pod 85 has an on/off button 84, an indicator light 88, and a plug port 86 for connecting a wire with the light-emitting appliance (not shown here). Battery pod 85 also has an unlock button 87. As seen in FIG. 6B, pressing unlock button 87 releases one end of neckband 82 for pulling out (see arrow A) to put it in open configuration so that the user can easily wear or remove neckband 82. To close and lock neckband 82, insert locking pin 83 of neckband 82 back into socket 81 on battery pod 85. Neckband 82 is made of soft plastic and is moderately flexible.

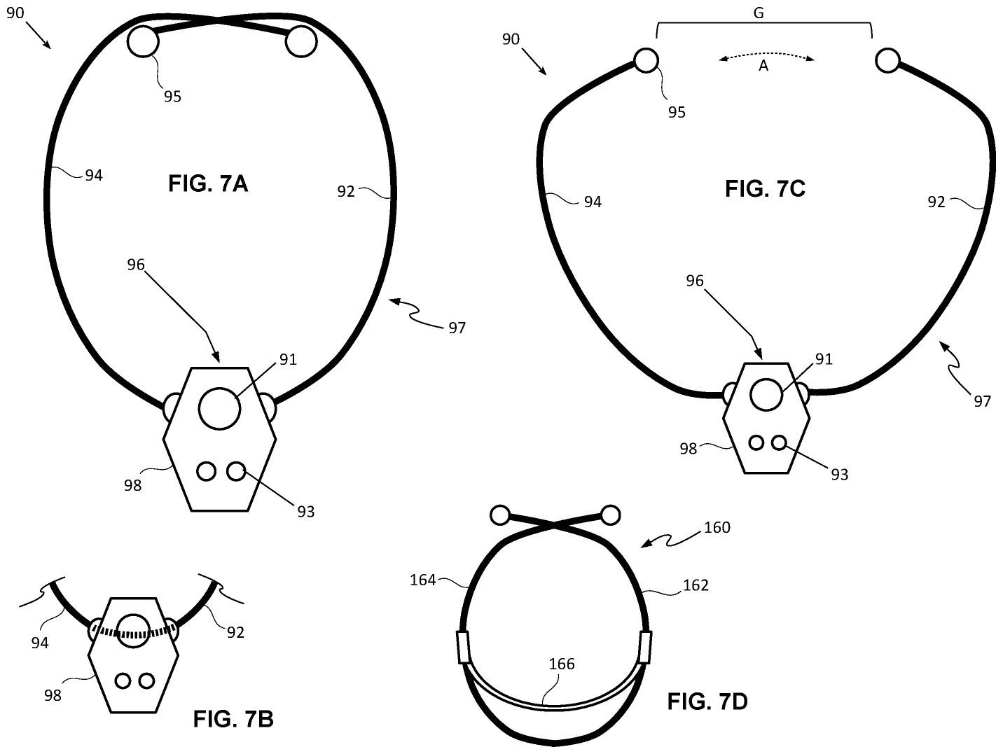

FIGS. 7A-7D show another example of a neckband assembly for the light therapy device. Neckband assembly 90 comprises a neckband 97 having two arms (left arm 92 and right arm 94) that are elastically flexible (elastically deformable) and its natural bending deflects inward. Arms 92 and 94 are made of metal. FIG. 7A shows neckband 97 in closed configuration with elastic flexible arms 92 and 94 in relaxed condition. In this closed configuration, a segment of arms 92 and 94 overlap (along a radial sweep) to close the loop around the user's neck. Neckband 97 carries a battery pod 98 for the battery compartment and user controls. Battery pod 98 has an on/off button 91, an indicator light 93, and a plug port 96 for connecting a wire with the light-emitting appliance (not shown here). Small beads 95 at the upper end of arms 92, 94 help improve user comfort.

FIG. 7B shows that arms 92 and 94 are actually a single continuous piece that travels through battery pod 98. Arms 92 and 94 do not have a fastening mechanism. In an alternate example, arms 92 and 94 are separate pieces that are separately connected to battery pod 98. To wear neckband 97, the user pulls arms 92 and 94 apart (see arrow A) to open the loop. FIG. 7C shows neckband 97 in open configuration. Arms 92, 94 are separated and not intersecting or overlapping (along a radial sweep). The user wears neckband 97 around the neck and then releases arms 92, 94 so that neckband 97 relaxes. Because of the natural inward bending of elastic arms 92 and 94, neckband 97 elastically reverts towards the closed configuration. Neckband 97 can now hang from the user's neck. After the treatment session, the user opens neckband 97 by pulling arms 92, 94 apart into open configuration and removes the neckband.

FIG. 7C also demonstrates how the gap (see bracket G) between the upper ends of arms 92 and 94 is measured (for some embodiments, which have narrow gap and wide gap configurations). For a neckband in narrow gap configuration, the gap distance G would be shorter than in wide gap configuration. FIG. 7D shows an alternate example of the neckband assembly (battery pod not shown) comprising a neckband 160 having elastically flexible arms 162 and 164. A leaf spring 166 is attached to arms 162 and 164 of the neckband to enhance elastic flexibility.

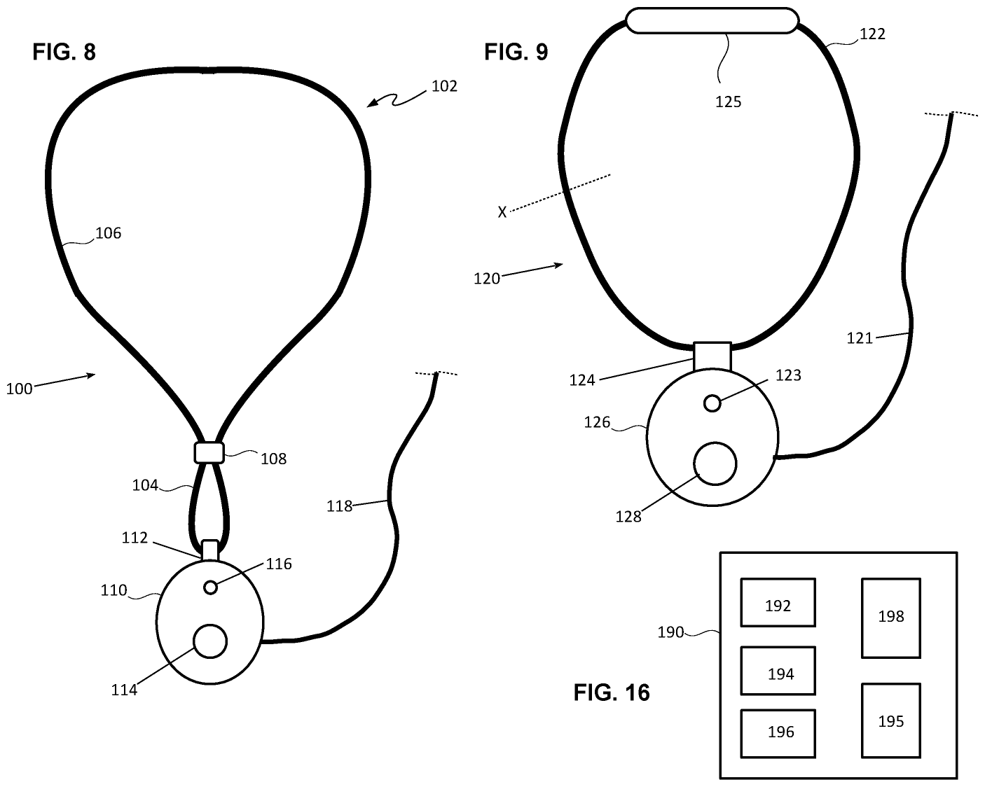

FIG. 8 shows another example of a neckband assembly for the light therapy device. Neckband assembly 100 comprises a loop neckband 102 that is in permanent closed loop configuration. Loop neckband 102 is made of soft plastic tubing. A sliding adjuster 108 allows the user to adjust the size of main loop 106 at the upper portion of neckband 102. This creates a loop tail 104 at the lower portion of neckband 102. In this example, the inner perimeter length (see below) of neckband 102 would include both main loop 106 and loop tail 104. Neckband assembly 100 further comprises a battery pod 110 attached to neckband 102 by a swivel hinge 112. Battery pod 110 has an activation button 114 and an indicator light 116. An electrical wire 118 connects battery pod 110 to supply power to the light-emitting appliance (not shown).

FIG. 9 shows another example of a neckband assembly for the light therapy device. Neckband assembly 120 comprises a loop neckband 122 that is in permanent closed loop configuration. Loop neckband 122 is made of soft plastic tubing. A soft padding 125 helps improve user comfort. Neckband assembly 120 further comprises a battery pod 126 that is attached to the neckband 122 by a bridge piece 124. Battery pod 126 has an activation button 128 and an indicator light 123. An electrical wire 121 connects battery pod 126 to supply power to a light-emitting appliance (not shown).

The size of the neckband for the therapy device could be characterized by its “inner perimeter length” for the neckband. This means the distance around the inner circumference of the neckband body. For neckbands that have an open and closed configuration, this is measured with the neckband in closed configuration. FIGS. 10A and 10B demonstrate how this definition is applied. FIG. 10A shows a closed loop neckband 130 in isolation. The inner perimeter length is measured along the inner circumference of neckband 130. Points on this path are indicated by the short arrows and traced by dashed line L1 (slightly offset from the inner circumference for better visibility). FIG. 10B shows an arc-shaped neckband 132 in isolation, which is an open loop with a discontinuous segment. Its inner perimeter length is measured along the inner circumference of neckband 132 (except for where it is discontinuous, that is, measured along the curved body only). Points on this path are indicated by the short arrows and traced by dashed line L2 (slightly offset from the inner circumference for better visibility).

The neckband for the therapy device could have any suitable inner perimeter length. For example, for neckbands in a closed loop configuration, the inner perimeter length could be 55 cm or 65 cm. For example, the neckband could have an inner perimeter length in the range of 55-150 cm or in the range of 65-150 cm. Note that this is greater than the girth for an average adult neck (neck circumference). See report by Joshipura et al, “Neck Circumference May Be a Better Alternative to Standard Anthropometric Measures” (2016) Journal of Diabetes Research, #6058916. In another example, for neckbands that do not form a fully closed loop (i.e. open loop such as an arc-shaped neckband), the neckband could have an inner perimeter length 45 cm or 60 cm. For example, the neckband could have an inner perimeter length of 45-120 cm or 60-120 cm.

FIG. 10B also shows how a neckband which does not have a fully closed loop configuration could be defined. Neckband 132 has a back upper segment 134 which is positioned at the back of the user's neck when worn by the user. Opposite upper/back segment 134, neckband 132 also has a front/lower side 136 positioned at the front or below the user's neck when worn. See that front/lower side 136 has a discontinuity in the loop formed by the body of neckband 132. Also, with the body of neckband 132 being absent here, there are no light diodes on the front lower side 136. These figures also demonstrate how a neckband could have the distinguishing characteristic of not having any light diodes for skin treatment on the inner perimeter side of the neckband.

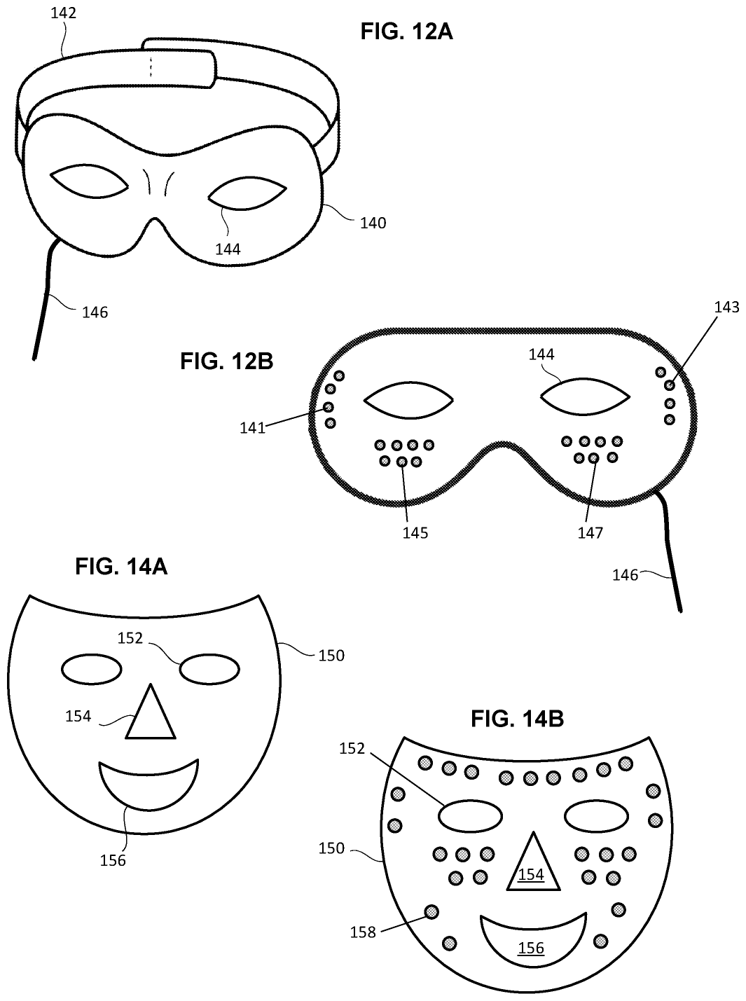

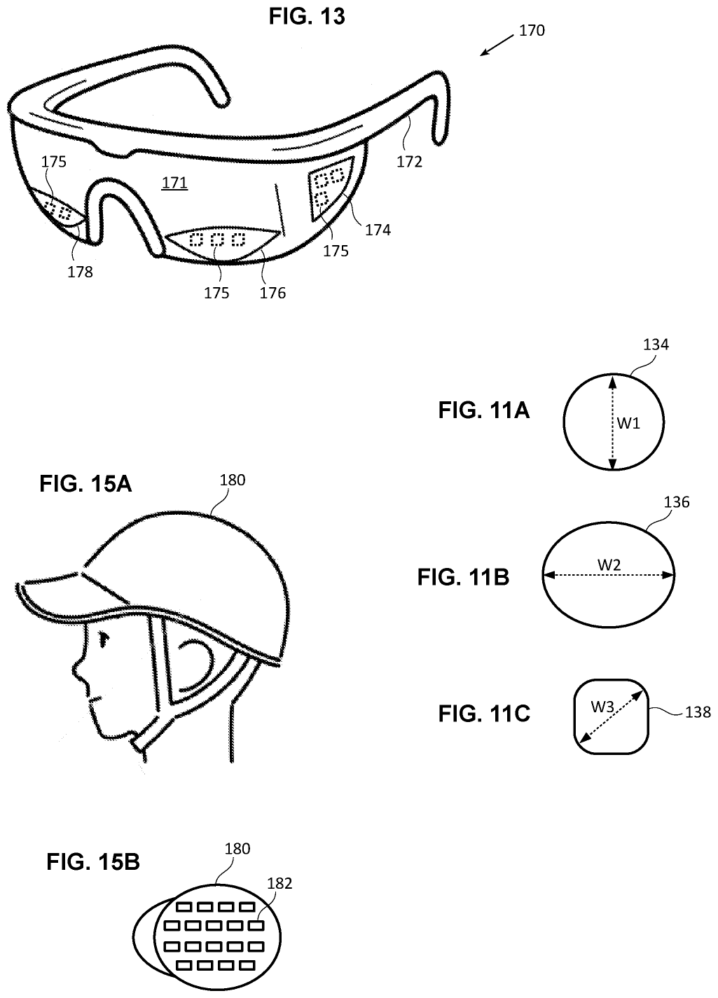

The size of the neckband for the therapy device could also be characterized by its maximum body width as measured on an imaginary transverse plane that cuts through the neckband at its point of maximum width. As examples of the transverse plane along the maximum body width, see cut lines X in FIG. 4 for neckband 32, FIG. 5B for neckband 55, and FIG. 9 for neckband 122. FIGS. 11A-11C demonstrate examples of how this definition is applied. FIG. 11A shows a transverse cross-section of a circle-shaped neckband body and its maximum body width W1, which is the diameter. FIG. 11B shows a transverse cross-section of an ovoid-shaped neckband body and its maximum body width W2, which is along the long axis. FIG. 11C shows a transverse cross-section of a square-shaped neckband body and its maximum body width W3, which is along the diagonal axis. The neckband for the therapy device could have any suitable maximum body width. For example, the maximum body width could be 5.0 cm or 3.5 cm. For example, the neckband could have a maximum body width in the range of 0.2-5.0 cm or in the range of 0.2-3.5 cm.

As another example of a light-emitting appliance, FIGS. 12A and 12B show an eyemask 140. FIG. 12A shows a front perspective view of eyemask 140 which comprises an elastic strap 142 and two eye holes 144. Eyemask 140 connects to the power supply of the device by a single electrical wire 146, which could be detachable or permanently connected. FIG. 12B shows the back side of eyemask 140 (facing towards the user) having four sets of LED lights: a set of LED lights 143 on the right lateral side, a set of LED lights 141 on the left lateral side, a set of LED lights 147 under right eye hole 144, and a set of LED lights 145 under left eye hole 144.

As another example of a light-emitting appliance, FIG. 13 shows an eyewear frame 170 that comprises two side arms 172 and a transparent eyescreen 171, which has multiple LEDs 175 that point inwards (towards the user's face). An undereye LED panel 176 is at the left bottom side of eyescreen 171 and another undereye LED panel 178 is on the right bottom side of eyescreen 171. Also, lateral LED panel 174 is on the left side of eyescreen 171 and another lateral LED panel (not shown) is on the right side of eyescreen 171.

As another example of a light-emitting appliance, FIGS. 14A and 14B show a full facemask 150 for covering the user's full face. FIG. 14A shows the front side of facemask 150, which has an elastic strap (not shown), two eye holes 152, a nose hole 154, and a mouth hole 156. FIG. 14B shows the back side of facemask 150 (facing towards the user), which has multiple LED lights 158 for treating various parts of the user's face. These LED lights are a mixture of blue and red color LEDs.

As another example of a light-emitting appliance, FIGS. 15A and 15B show a helmet 180 for hair growth. FIG. 15A shows the user wearing helmet 180 on top of their head. FIG. 15B shows the underside of helmet 180, which has an array of laser diodes 182. Laser diodes 182 emit red light to promote hair growth on the user's scalp.

FIG. 16 shows an example of a skin therapy kit 190 as a block diagram. Kit 190 comprises the following items: a light-emitting appliance 192; a neckband assembly 194 comprising a neckband and battery pod (which contains a rechargeable battery); an electrical wire 196 for connecting the battery pod to the light-emitting appliance 192; a bottle of collagen peptides 198 for oral administration; and a vial of hyaluronic acid 195 as a topical skin cream. Using collagen peptides 198 and hyaluronic acid 195 together with the light therapy device may enhance the benefits of light therapy.

The foregoing description and examples merely illustrate the invention and are not intended to be limiting. Each of the disclosed aspects and embodiments of the invention may be considered individually or in combination with other aspects, embodiments, and variations of the invention. Also, unless otherwise specified, the steps of the methods of the invention are not limited to any particular order of performance. Persons skilled in the art may perceive modifications to these embodiments that incorporate the spirit and substance of the invention. Such modifications are within the scope of the invention.

Any use of the word “or” herein is intended to be inclusive and is equivalent to the expression “and/or,” unless the context clearly indicates otherwise. As such, for example, the expression “A or B” means A, or B, or both A and B. Similarly, for example, the expression “A, B, or C” means A, or B, or C, or any combination thereof.

1. A light therapy device for treating a user's skin, comprising:
a neckband for wearing on the user's neck;
a power supply within the neckband or carried by the neckband, wherein the power supply is a battery or battery compartment for receiving a battery;
a light-emitting appliance for applying to a part of the user's head, wherein the light-emitting appliance comprises light diodes;
an external electrical wire for connecting the power supply to the light-emitting appliance.

2. The device of claim 1, wherein the neckband is part of a neckband assembly comprising the neckband and the power supply, and the neckband carries the power supply.

3. The device of claim 2, wherein the power supply is a battery pod that comprises a battery or a battery compartment for receiving a battery.

4. The device of claim 2, wherein the neckband comprises two arms that meet and separate by a releasable fastener mechanism.

5. The device of claim 1, wherein the neckband has an inner perimeter side, and the neckband does not have any light diodes on the inner perimeter side.

6. The device of claim 1, wherein the neckband has an open configuration and a closed configuration.

7. The device of claim 6, wherein the neckband comprises a releasable fastener mechanism that is engaged in the closed configuration and disengaged in the open configuration.

8. The device of claim 2, wherein the neckband has a permanent closed loop configuration.

9. The device of claim 1, wherein the electrical wire is pre-connected to the light-emitting appliance, or is pre-connected to the power supply, or is a separate component for connection to both the light-emitting appliance and the power supply.

10. The device of claim 1, wherein the length of the electrical wire is 75 cm.

11. A light therapy device for treating a user's skin, comprising:
a neckband assembly comprising:
a neckband for wearing on the user's neck;
a battery pod carried by the neckband, wherein the battery pod comprises a battery or a battery compartment for receiving a battery;
a light-emitting appliance for applying to a part of the user's head, wherein the light-emitting appliance comprises light diodes;
an external electrical wire for connecting the battery pod to the light-emitting appliance.

12. The device of claim 11, wherein the neckband has an open configuration and a closed configuration.

13. The device of claim 12, wherein the neckband comprises two arms, and wherein the neckband is in closed configuration when the two arms meet and in open configuration when the two arms are separated.

14. The device of claim 13, wherein the two arms meet by a releasable fastener mechanism.

15. A method for skin therapy, comprising:
having a light therapy device of claim 1;
wearing the neckband on the neck;
applying the light-emitting appliance to a treatment site on the head;
activating the device to cause the light-emitting appliance to emit therapeutic light;
performing light therapy to the treatment site;
removing the neckband from the neck.

16. The method of claim 15, further comprising connecting the external electrical wire to the power supply, or the light-emitting appliance, or both.

17. The method of claim 15, wherein the neckband has an open configuration and a closed configuration, and the method further comprises:
having the neckband in open configuration to wear the neckband;
closing the neckband into closed configuration;
after performing the light therapy, opening the neckband into open configuration to remove the neckband.

18. The method of claim 17, further comprising opening the neckband into open configuration to wear the neckband.

19. The method of claim 17, wherein the neckband has a releasable fastener mechanism, and the method further comprises:
having the fastener mechanism disengaged to wear the neckband;
engaging the fastener mechanism to close the neckband;
disengaging the fastener mechanism to open the neckband for removal.

20. The method of claim 19, further comprising disengaging the fastener mechanism to open the neckband for wearing.
